(19) United States
(12) Patent Application Publication
(10) Pub. No.: US 2003/0211636 A1
(43) Pub. Date: Nov. 13, 2003

(54) Point of care test for measurement of therapeutic drug levels

(75) Inventors: Vahe Bedian, East Lyme, CT (US); Stafford McLean, Stonington, CT (US); Ronald S. Obach, Gales Ferry, CT (US); Holly D. Soares, Noank, CT (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/418,822

(22) Filed: Apr. 18, 2003

Related U.S. Application Data
(60) Provisional application No. 60/373,786, filed on Apr. 19, 2002.

(51) Int. Cl. G01N 33/543

(52) U.S. Cl. 436/518; 436/111

(57) ABSTRACT

The present invention provides methods of determining the proper dosage of Compound 122 (and other drugs that are metabolized by cytochrome P450 2D6) to be given to a patient. Also provided are methods of determining the metabolizer status of persons, devices for performing the invention methods, and antibodies for use in these devices and methods.

### RELATED APPLICATION

This application claims priority of U.S. application No. 60/373,786 filed Apr. 19, 2002.

### FIELD OF THE INVENTION

The present invention relates to a simple test for evaluating the status of a potential patient with respect to their ability to bioassimilate and metabolize (2-methoxy-5-trifluoromethoxy-benzyl)-(2-phenyl-piperidin-3-yl)-amine (hereinafter referred to as Compound 122) and other compounds that are metabolized primarily by cytochrome P450 2D6. The results of this assay are useful for determining optimal dosing of such compounds when given to the patient to treat an illness.

### BACKGROUND OF THE INVENTION

It is well known that the effect of a drug will be related to the concentration of the drug, and active metabolites of that drug should they exist, in the body. The concentrations of many drugs need to be within a certain range; high enough to elicit the intended effect yet not so high as to cause unwanted side effects or toxicities. The human population is heterogeneous with regard to the rates and mechanisms involved in determining what these drug concentrations will be. This heterogeneity can be due to both genetic and environmental factors. Pharmacokinetic aspects of drugs that can be effected by this heterogeneity include the rate and extent of oral absorption, the extent of first-pass hepatic extraction, volume of distribution, clearance, and half-life. This heterogeneity can confound treatment, since the same dose of any given drug can be efficacious in one subject, without any effect in a second subject, and toxic in a third. Presently, physicians have no means by which to tell how a patient will respond pharmacokinetically to a drug. If physicians could test patients prior to starting therapy, to gain prospective information on how a patient will respond to a drug, therapy overall will be improved. There is a present need for rapid, simple, non-invasive, convenient, and accurate tests for determination of drugs in biological fluids, especially tests that could be used conveniently and rapidly in a physician's office or by a patient at home.

The cytochrome P450 family of enzymes is primarily responsible for the metabolism of xenobiotics such as drugs, carcinogens, and environmental chemicals, as well as several classes of endobiotics such as steroids and prostaglandins. Members of the cytochrome P450 family are present in varying levels and their expression and activities are controlled by variables such as chemical environment, sex, developmental stage, nutrition, and age.

More than 200 cytochrome P450 genes have been identified. There are multiple forms of these P450 and each of the individual forms exhibit degrees of specificity towards individual chemicals in the above classes of compounds. In some cases, a substrate, whether it be drug or carcinogen, is metabolized by more then one of the cytochromes P450. Genetic polymorphisms of cytochromes P450 result in phenotypically distinct subpopulations that differ in their ability to perform biotransformations of particular drugs and other chemical compounds.

These phenotypic distinctions have important implications for selection of drugs. For example, a drug that is safe when administered to most humans may cause toxic side-effects in an individual suffering from a defect in an enzyme required for detoxification of the drug. Alternatively, a drug that is effective in most humans may be ineffective in a particular subpopulation because of lack of a enzyme required for conversion of the drug to a metabolically active form. Further, individuals lacking a biotransformation enzyme are often susceptible to cancers from environmental chemicals due to inability to detoxify the chemicals (Eichelbaum et al., Toxicology Letters, 64165:155-22 (1992)). Accordingly, it is important to identify individuals who are deficient in a particular P450 enzyme, so that drugs known or suspected of being metabolized by the enzyme are not used, or used only with special precautions (e.g., reduced dosage, close monitoring) in such individuals. Identification of such individuals may indicate that such individuals be monitored for the onset of cancers.

Cytochrome P450 2D6, also known as debrisoquine hydroxylase, is the best characterized polymorphic P450 in the human population (Gonzalez et al., Nature, 331 :442-46 (1988)). A poor metabolizer phenotype has been reported which behaves as an autosomal recessive trait with an incidence between 5 and 10% in the white population of North America and Europe. Poor metabolizers exhibit negligible amounts of cytochrome P450 2D6 (Gonzales et al., supra). Genetic differences in cytochrome P450 2D6 may be associated with increased risk of developing environmental and occupational based diseases. See Gonzalez & Gelboin, J. Toxicology and Environmental Health, 40:289-308 (1993)).

Several drugs for treating cardiovascular and psychiatric disorders are known substrates of cytochrome P450 2D6 (Dahi and Bertilsson, Pharmacogenetics, 3:61-70 (1993)), a situation that creates problems in prescribing such drugs. Although such drugs may be the most effective treatment for most of the population, physicians are reluctant to prescribe them due to the risk of adverse effects in poor metabolizers (Buchert et al., Pharmacogenetics, 2:2-11 (1992); Dahl et al., Pharmacogenetics, 3:61-70 (1993)).

For more information on cytochrome P450 2D6 and methods of dealing with poor metabolizers, see also U.S. Pat. No. 6,060,253.

Compound 122, a new drug potentially useful for many indications, has been shown to be metabolized by at least two enzymes: cytochromes P450 2D6 and P450 3A4. Those individuals devoid of functional CYP2D6 activity are termed poor metabolizers (PMs) and are at risk to greater drug exposure than those individuals with one or more functional copies of the CYP2D6 gene. Those individuals with more typical levels of CYP2D6 activity are called extensive metabolizers (EMs). CYP2D6 can also be subjected to inhibition by other drugs (e.g., quinidine and paroxetine) and patients taking such agents will exhibit lower CYP2D6 activity. As a result, genotyping for CYP2D6 alone will not always identify patients at risk of greater exposure. Due to the inter-patient variability in CYP2D6 and CYP3A4 activities, at a specified dosing regimen, Compound 122 exposures can vary considerably among the population. A device that could measure Compound 122 concentrations in biological fluids would be of assistance in optimizing therapy with this compound, by customizing the dosing regimen for each patient to deliver specified exposures.

Over the past 10 years, many in vitro diagnostic test kits have been commercialized that utilize the principles of immunochromatography. The first major target analyte for this test format was (human) Chbrionic Gonadotropin (hCG) for the detection of pregnancy. Pregnancy kits have been developed that use urine or plasma as test solutions; that use latex, selenium, or gold conjugates as detector reagents; that require as little as 90 seconds or as much as 15 minutes to perform, and that have readout zones which may consist of a single bar for a positive reaction (i.e., sample containing more than 25 mIU/mL of hCG), or two bars for a positive reaction (one bar in this case would indicate a negative reaction). Many of the tests are equipped with a zone at the end of the test strip that will change color when the sample front reaches it, thereby telling the user that the test is complete and that it is time to interpret the results (end of assay).

In addition to the impressive array of commercially available pregnancy tests, there are also test strip assays on the market for Streptococcus (Strep-A), Luteinizing Hormone (LH) and Estradiol (E2) for ovulation prediction, Malaria and a variety of other tropical infectious diseases, Hepatitis B (antigen and antibody), Hepatitis C, Hemoglobin, HIV (antibody); Heliobacter pylori (H. pylori, ulcer detection); Troponin (cardiac monitoring); and for a range of different drugs of abuse. There are probably products available for other analytes that haven't been listed and new ones that will become available in the near future. The majority of the analytes listed above are measured on the basis of presence/absence (yes/no), and most of them are detected using immunometric assays. For immunometric-type assays, a ligand specific for the analyte (normally, but not necessarily an antibody [Ab]) is immobilized to the membrane. The detector reagent, typically an antibody coupled to latex or colloidal metal, is deposited (but remains unbound) into the conjugate pad. When sample (urine, plasma, whole blood, etc.) is added to the sample pad, it rapidly wets through to the conjugate pad and the detector reagent is solubilized. The detector reagent begins to move with the sample flow front up the membrane strip. Analyte that is present in the sample will be bound by the antibody that is coupled to the detector reagent. As the sample passes over the zone to which the capture reagent has been immobilized, the analyte detector reagent complex is trapped. Color develops in proportion to the amount of analyte present in the sample.

There are also commercially available assays for drugs of abuse and for steroid-based ovulation prediction that are based on competitive immunoassay protocols. In this type of assay, the detector reagent is typically the analyte (or an analog of the analyte) bound to latex or a colloidal metal. As the sample (containing analyte) and detector reagent pass over the zone to which the capture reagent (typically an antibody) has been immobilized, some of the analyte and some of the detector reagent are bound and trapped. The more analyte present in the sample, the more effectively it will be able to compete with, and/or displace, the binding of detector reagent. The hallmark of most competitive immunoassays is that an increase in the amount of analyte in the sample results in a decrease of signal in the readout zone.

A very useful reference for production of immunochromatographic test strips is Millipore's Short Guide For Developing Immunochromatographic Test Strips (2nd Edition, 1999). This document is most easily accessed through Millipore's website at www.millipore.com. Other useful references include U.S. Pat. Nos. 5,238,652 and 6,194,221.

### SUMMARY OF THE INVENTION

The present invention is directed to methods and reagents used to detect levels of Compound 122 in bodily fluids. The method uses ELISA, RIA, chemiluminescence, immunofluorescent, lateral flow and flow-through immunochromatographic techniques. In addition, the invention will utilize antibodies selective for parent compound, antibodies that detect the desmethyl metabolite of parent compound, chemically tagged compound and chemically tagged desmethyl metabolite to detect Compound 122 in bodily fluids utilizing both direct and competitive immunochemical bioassays. Bioassay formats consist of standard plate-based immunoassays and self-contained devices designed to be read by an unskilled operator. The invention further relates to packaged items for the immunochromatographic kits as well as to novel reagents in the test devices utilized to detect Compound 122 levels. The information gained by use of the bioassays will be for the assessment of response of subjects to Compound 122 and/or other drugs, with regard to exposure, and adjustment of dose, as necessary, to target desired exposure values.

In a first aspect, the present invention provides a method for determining whether a person is a poor metabolizer or an extensive metabolizer of Compound 122, said method comprising the steps of administering a test dosage of Compound 122 to said person, measuring the concentration of Compound 122 in a saliva sample from said person at a predetermined time period after said administration step, and classifying said person as a poor metabolizer or an extensive metabolizer of Compound 122 based upon the concentration of Compound 122 as measured in said measuring step.

In a second aspect, the present invention provides a method for determining the proper dose of Compound 122 to be given to a patient, said method comprising the steps of administering a test dosage of Compound 122 to said patient, measuring the concentration of Compound 122 in a saliva sample from said patient at a predetermined time period after said administration step, and determining that said patient requires a low dosage treatment of Compound 122 if said saliva concentration is high, and that said patient requires a standard dosage treatment of Compound 122 if said saliva concentration is low.

In a third aspect, the present invention provides a device for measuring the salivary concentration of Compound 122, said device comprising a lateral flow membrane, a saliva application zone on said membrane, and an indicator zone on said membrane spaced laterally from said saliva application zone; wherein immobilized in said indicator zone is an antibody specific for Compound 122, which antibody is labeled in a manner that provides an easily read color change if saliva applied to said saliva application zone contains an adequate concentration of Compound 122.

In a fourth aspect, the present invention provides an antibody that specifically binds to Compound 122 wherein said antibody does not specifically bind to naturally occurring metabolites of Compound 122.

### DESCRIPTION OF THE DRAWINGS

FIG. 1 provides chemical structures of the antigens utilized to generate antibodies for use in the invention methods.

FIG. 2 provides exemplary immunochromatographic formats of self-contained bioassay.

FIG. 3 provides a graphical representation of concentrations of Compound 122 measured by HPLC-MS/MS-based analytical methods in saliva and serum of healthy subjects receiving doses of Compound 122 at a specific time post-dose. This graph demonstrates that the salivary concentrations are predictive of circulating concentrations attained after continued dosing.

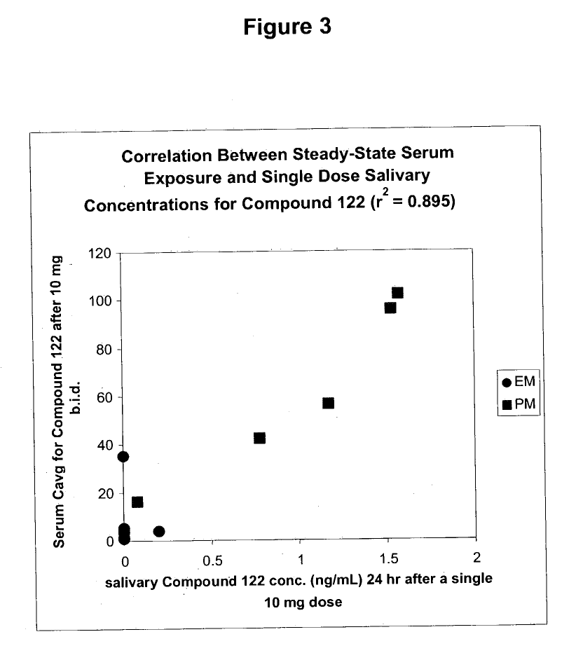

### DETAILED DESCRIPTION

The present invention is primarily useful for determining whether a given patient, which patient would benefit by treatment with Compound 122, should be given a low dosage treatment or a standard dosage treatment of Compound 122. Because Compound 122 is primarily metabolized by cytochrome P450 2D6, and because a significant fraction of the population has little or no activity of such enzyme, it is important to treat those who poorly metabolize Compound 122 with low dosage treatment in order that they are not overdosed therewith, and do not suffer unduly from any potential side effects thereof. However, it is also possible to estimate a patient's ability to metabolize Compound 122 based upon their metabolization of other compounds that are metabolized primarily by 2D6. Such compounds include dextromethorphan and others known to those skilled in the art. However, since Compound 122 is also metabolized by cytochrome P450 3A4, use of other compounds as test compounds is not without risk. In some cases a person may be a poor metabolizer of one of Compound 122 versus another test compound, and an extensive metabolizer of the other. Conversely, it is also possible, subject to the same risks, to use Compound 122 as a test compound to classify a patient as a poor or extensive metabolizer with respect to 2D6, and use this classification to make a dosage determination for another 2D6 metabolized drug. Thus, the methods of the present invention, even when Compound 122 is used as the test compound, may be applied to dosing regimens for drugs other than Compound 122 itself.

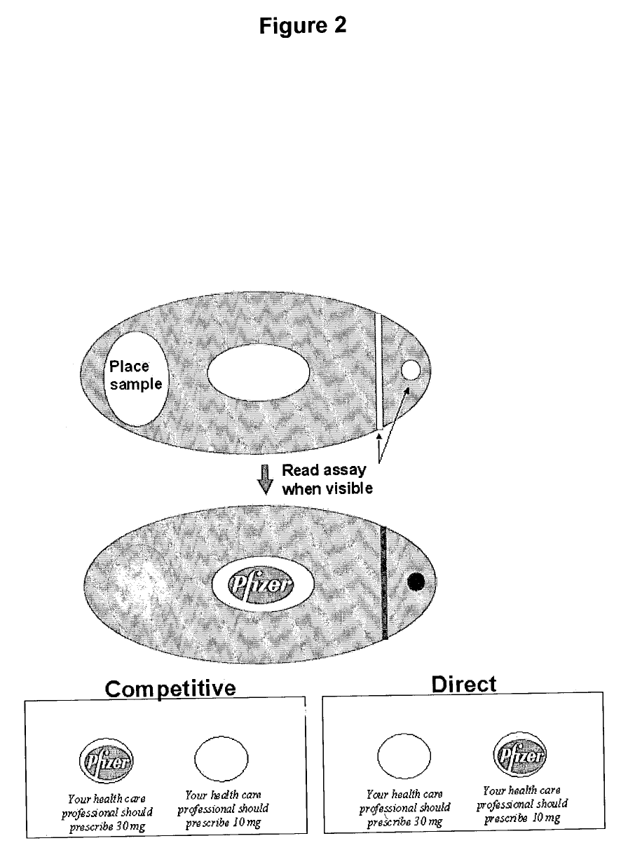

In preferred embodiments, Compound 122 is administered to the patient in a doctor's office or at home, and the saliva sample taken soon thereafter. The device used will preferably be comparable to the home pregnancy test kits commonly available today, and as such the patient him or herself will be able to easily collect the saliva sample and determine the test result. See FIG. 2 for exemplary devices. Such “dipstick” assays are already well known to the public, and are easily used with very simple instructions. Even with untrained users, the error rate due to user error can be made very low.

In order to detect the antibody/antigen complex within the assay device, a detector reagent must be coupled to the antibody or antigen. Exemplary detector reagents include colored latex particles, colloidal metal, enzyme, and the like. All of these and more are commercially available from a variety of well known companies.

Different types of equipment will be required to produce components and finished (prototype and manufactured) product. Manufacturing steps that typically require specialized equipment include applying reagents onto or into membranes, sample pads, reagent pads, and other porous media; laminating membranes, sample pads, conjugate pads, and absorbent pads onto a support backing so that a precise overlap between each of the porous media is created; cutting sheets or rolls into strips of defined length and width; and assembling test strips (picking and placing) into plastic housings.

The polymer from which the membrane is made will determine most of the membrane's binding characteristics. Certain post-treatments (e.g., coating with high levels of polyvinylpyrrolidone) and the addition of secondary polymers (e.g., Millipore's patented hydrophilization process) may dramatically alter the ability of a particular membrane polymer (e.g., nitrocellulose, polyvinylidene fluoride, Teflon) to bind protein.

For the most part, a membrane's protein binding capacity is determined by the amount of surface area available for immobilization. A membrane's surface area is determined by its pore size, porosity (amount of air in the three dimensional structure), thickness, and to a minor extent, by structural characteristics unique to the polymer from which it is made. All other parameters being equal, surface area decreases with increasing pore size (non-linear), increases with increasing thickness (linear), and increases with increasing porosity (non-linear).

FIG. 2 provides some exemplary lateral flow or similar devices. The upper portion of FIG. 2 shows a cutaway side view of a typical lateral flow or dipstick device. The portion labelled A is the region for body fluid application or the wicking region. The portion labelled B is the conjugate pad containing colored detector and control reagents. Detector reagent is resuspended upon absorption of fluid front. The portion labelled C is the nitrocellulose membrane that carries the fluid front by capillary action. The portion labelled D is the capture region containing permanently immobilized capture reagents. The portion labelled E is the secondary capture reagent for control substance. The portion labelled F is the absorbance pad. Color in E and F indicate successful use of device and end of read.

The lower portions of FIG. 2 show schematics of device and outcomes based upon competitive or direct format.

In addition to lateral flow technologies, there are other detection means that are suitable for point-of-care use that are known to those skilled in the art. For example, Up-converting Phosphor Technology (UPT) is a relatively new reporter system that converts low energy infra-red to high energy visible light. The reporter can be applied to any solid surface include membranes, particle beads and antibodies to be used for detection of proteins and nucleic acids. The advantage is improved sensitivity with very little background noise in the point-of-care format as well as simultaneous detection of multiple antigens. In addition, the signal does not fade with time, the platform is amenable to miniaturization, and the reporter is useful in any matrix. For more information see Ziljlams et al., Anal. Biochemistry, 267(1):30-36 (1999).

Another set of alternatives to lateral flow devices are methods that use DNA amplification of protein signal via a microfluidic platform or micro total analysis systems (TAS) and strand displacement amplification (SDA). Other POC technologies take advantage of microfluidic chip design that enables using nanoscale quantities of reagents. The ideal is that the entire sample handling and detection process takes place on a biochip specifically designed for that purpose. Microfluidics is typically applied to POC DNA amplification, but can be applied to protein based detection. In brief, the antibody can be tagged with a DNA probe that is then amplified by various microfluidic strategies. Extraction and amplification all take place on specialized chip. Handylab (Ann Arbor, Mich.) is a leader in this type of technology. See also Yang et al., Biosensors and Bioelectronics, 17(6-7):605-618 (2002).

Generation and Characterization of Antibody: The therapeutic or diagnostic usefulness of a monoclonal antibody (MAb) is dependent upon several factors. The MAb must possess sufficient binding affinity and a relatively high avidity for an antigen. The avidity of a MAb is based on the valency of the antibody (and the antigen) and the quaternary arrangement of the interacting components. To be useful, MAbs need to be specific enough to distinguish between levels of parent and metabolite compounds. The difficulty is identifying/producing antibodies which possess sufficient affinity, avidity, and selectivity to be useful in detecting low levels of small drug molecules in bodily fluids.

The antibodies may be obtained by immunizing an animal with a small drug molecule conjugate that is comprised of the target molecule (e.g., Compound 122) conjugated to BSA or KLH using commercially available cross-linking reagents, or biotinylated target molecule complexed with avidin. In addition to these specific examples, other means of increasing the immunizing character of a small molecule are known to those skilled in the art. The RIMMS protocol described by Kilpatrick, et al. (Hybridoma, 16:381-389 (1997)) or conventional splenocytes fusions (see methods in “Monoclonal Antibodies”, R. Kennett, ed. Plenum Press (1980)) may be used. Briefly, the RIMMS procedure uses 5-6 immunizations directed towards draining lymph nodes within a two week period, followed by harvest of lymph node lymphocytes. Conventional immunizations use a 4-8 week immunization scheme, followed by harvest of the spleen and isolation of splenocytes. Both methods may use Freund's, Ribi, TiterMax, CpG DNA, or alum as adjuvant.

To achieve specific recognition of target molecule versus a metabolite, or vice versa, immunosuppression with cyclophbsphamide may be used. In this procedure negative antigen is first injected at appropriate dose With 100 mg/kg of weight cyclophosphamide (CP, freshly made from Sigma 1 g Isopacks, discard after 1 week) but without adjuvant. Additional 100 mg/kg doses of CP is administered at 24 and 48 hrs after first injection. Animals are allowed to recover for 7-10 days, then normal immunization schedule with positive antigen is started. Following harvest of lymph node and/or splenic lymphocytes, hybridomas may be produced by PEG fusion procedures (e.g., see Example 2). mAbs can also be prepared by phage display, cloning of cDNAs or other molecular biological techniques known in the art. Hybridomas may be screened by ELISA (see Examples 3 and 4), though again, other techniques may be selected by those skilled in the art.

mAbs selected as target molecule or metabolite specific by ELISA may be further characterized for affinity by surface plasmon resonance using a BIAcore 2000, using procedures known in the art. Briefly, purified antibodies may be immobilized to the surface of a BIAcore chip, and the binding of the target molecule or metabolite may be analyzed in terms of on and off rates, and equilibrium dissociation and affinity constants. Conversely, the small molecule drugs or biotinylated derivatives can be captured on the BIAcore chip, and the binding kinetics and affinity of antibodies can be determined.

To perform the invention methods herein, the patient must first be given a test dosage of a compound metabolized primarily by 2D6. In the most preferred embodiments, the compound is Compound 122. The amount needs to be enough to produce a detectable amount of Compound 122 in the saliva of poor metabolizers, and not so much that harmful side effects might be produced. Amounts of Compound 122 from 2 mg up to 100 mg are feasible, with 10-30 mg being preferred, and 10 mg being the presently most preferred amount.

Test dosages may be administered to the patient in any convenient manner, with oral administration being most preferred. Those of skill in the art are aware of multitudinous other options.

In order to standardize the methods, it is important that the saliva sample be collected at a predetermined time interval after administration. This time interval can vary from 0.5 hours up to 24 hours, with 1 to 4 hours being preferred, and 2 hours being the presently most preferred period.

Based upon the present research, it is shown that the amount of Compound 122 in a patient's saliva a few hours after a single dose of Compound 122 correlates positively with the amount of Compound 122 present in a patient's serum after many days of daily dosing. A single dose of Compound 122 achieves salivary concentrations of between about 0.01 and 0.9 ng/ml in extensive metabolizers, and more often concentrations between about 0.1 and 0.5 ng/ml, when measured a few hours after a 10-30 mg dose. Contrarily, a single dose of Compound 122 achieves salivary concentrations of between about 1.1 and 5.0 ng/ml in poor metabolizers, and more often concentrations between about 2.0 and 3.0 ng/ml, when measured a few hours after a 10-30 mg dose. Thus, 1.0 ng/ml is the preferred cutoff concentrations for distinguishing poor metabolizers from extensive metabolizers of Compound 122. However, if significantly greater or smaller test dosages of Compound 122 are given, or if the time interval between test dosage administration and saliva collection are changed, then a different cutoff concentration will likely be necessary for distinguishing poor metabolizers from extensive metabolizers.

The saliva sample that is collected is preferably measured immediately upon collection. Storage is possible, but not preferred. In a most preferred embodiment, the same device that is used for collecting the saliva is the measurement device itself, i.e., a lateral flow assay or dipstick device that collects the saliva by absorption into a collection area and wherein the saliva flows through the device from the application area to the indicator area. However, if more exact measurements are necessary, laboratory methods, such as those described in Example 6, are possible.

Those skilled in the art will fully understand the terms used herein in the description and the appendant claims to describe the present invention. Nonetheless, unless otherwise provided herein, the following terms are as described immediately below.

By “low dosage treatment” is meant daily dosages totaling less than or equal to about 20 mg of Compound 122.

By “standard dosage treatment” is meant daily dosages totaling more than or equal to about 30 mg of Compound 122.

By “high saliva concentration of Compound 122” is meant concentrations exceeding about 1 ng/ml when measured a few hours after test dosage administration.

By “low saliva concentration of Compound 122” is meant concentrations below about 1 ng/ml when measured a few hours after test dosage administration.

By “naturally occurring metabolites of Compound 122” is meant those metabolites of Compound 122 that are produced when Compound 122 is administered to a human patient.

By “lateral flow device” is meant a device that absorbs or adsorbs a liquid sample, routes that liquid sample to a detection zone, and uses antibody-based detection methods to generate a visible signal in response to the presence or absence of a specific antigen.

Other features and advantages of the invention will be apparent from the following detailed description and from the claims. While the invention is described in connection with specific embodiments, it will be understood that other changes and modifications that may be practiced are also part of this invention and are also within the scope of the appendant claims. This application is intended to cover any equivalents, variations, uses, or adaptations of the invention that follow, in general, the principles of the invention, including departures from the present disclosure that come within known or customary practice within the art. Additional guidance is found in standard textbooks of molecular biology, protein science, immunology, and the like. All publications cited in this document are herein incorporated by reference in their entirety.

### EXAMPLES

### Example 1

### Generation of Antigens

Synthesis of Antigen 1: N_{piperidino}-succinyl Compound 122

19.0 mg of Compound 122 (50 μmol) and 5.0 mg succinic anhydride (50 μmol) were dissolved in 0.4 mL acetonitrile and 0.05 mL of triethylamine. The reaction was mixed at 50° C. for 90 minutes then purified on reverse phase HPLC (Vydac C4 column) using a water/acetonitrile gradient. Yield: 19.4 mg (79%). LC-ESMS monoisotopic m/z for MH+=482.2 (expect 482.2). Previous experiments from these labs indicated that Compound 122 reacts with acylating agents preferentially at the piperidino nitrogen. In the present case, succinylation of N_{piperidino} was confirmed by 2D HNMR experiments.

Synthesis of Antigen 2: N_{piperidino}-succinyl-2-[(2-phenyl-piperidin-3-ylamino)-methyl]-4-trifluoromethoxy-phenol

18.4 mg of 2-[(2-phenyl-piperidin-3-ylamino)-methyl]-4-trifluoromethoxy-phenol (50 μmol) and 5.0 mg succinic anhydride (50 μmol) were dissolved in 1.0 mL acetonitrile and 0.05 mL of triethylamine. The reaction was mixed at 50° C. for 180 minutes then purified on reverse phase HPLC (Vydac C4 column) using a water/acetonitrile gradient. Yield: 8 mg (34%). LC-ESMS monoisotopic m/z for MH+=467.2 (expect 467.2).

Synthesis of Antigen 3: Biotinyl-diaminodiethylene Glycol-N_{piperidino}-succinyl Compound 122

4.8 mg of antigen 1 (10 μmol) was dissolved in 0.2 mL DMF. To this was added in sequence: 1.4 mg HOAt (1-hydroxy-7-azabenzotriazole), 3.8 mg HATU (O-(7-azabenzotriazol-1-yl)-N,N,N′,N′-tetramethyluronium hexafluorophosphate; 10 μmol), and 10 μL DIEA. The solution was mixed for 2 mins then 8.4 mg of Biotin PEO-LC-Amine (Pierce product # 21347) predissolved in 0.1 mL DMF was added. After 5 minutes, analytical HPLC showed that the reaction was complete. The mixture was purified on reverse phase HPLC (Vydac C4 column) using a water/acetonitrile gradient. Yield: 5.0 mg (57%). LC-ESMS monoisotopic m/z for MH+=881.4 (expect 881.4).

Synthesis of Antigen 4: Biotinyl-diaminodiethylene Glycol-N_{piperidino}-succinyl Compound 122

4.7 mg of antigen 2 (10 μmol) was dissolved in 0.2 mL DMF. To this was added in sequence: 1.4 mg HOAt, 3.8 mg HATU (10 μmol), and 10 μL DIEA. The solution was mixed for 2 mins then 8.4 mg of Biotin PEO-LC-Amine (Pierce Product # 21347) predissolved in 0.1 mL DMF was added. After 5 minutes, analytical HPLC showed that the reaction was complete. The mixture was purified on reverse phase HPLC (Vydac C4 column) using a water/acetonitrile gradient. Yield: 2.2 mg (25%). LC-ESMS monoisotopic M/z for MH+=867.6 (expect 867.4).

Conjugation of Antigen 1 and Antigen 2 to Bovine Serum Albumin (BSA)

2.0 mg of Imject BSA (Pierce) suspended in 0.2 mL sterile water was mixed with 2.0 mg of Antigen 1 and 0.2 mL conjugation buffer (Pierce Imject EDC Conjugation Buffer Catalog # 77162). 1.0 mg EDC (ethyl dimethylaminopropyl carbodiimide, Pierce) was added to the solution which was allowed to react for 2 hour at 23° C. then at 4° C. overnight. The low molecular weight reagents were removed by passing the solution over a 5 mL polyacrylamide 6000 desalting column (Pierce). 1.0 mL fractions were collected, the protein containing fractions were identified by UV absorbance at 280 nm, and pooled. Covalent conjugation was confirmed by MALDI-TOF analysis, which showed an average mass gain of 3,000 Da (about 8 molecules of Antigen 1 per molecule of BSA). A similar average mass gain was observed for Antigen 2.

Conjugation of Antigen 1 and Antigen 2 to Keyhole limpet hemocyanin (KLH)

10 mg of Imject KLH (Pierce) suspended in 0.2 mL sterile water was mixed with 2.0 mg of Antigen 1 and 0.2 mL conjugation buffer. 1.0 mg EDC was added to the solution, which was allowed to react for 2 hr at 23° C. then at 4° C. overnight. The low molecular weight reagents were removed by passing the solution over a 5 mL polyacrylamide 6000 desalting column (Pierce). 1.0 mL fractions were collected, the protein containing fractions were identified by UV absorbance at 280 nm, and pooled. Antigen 2 was treated similarly. MALDI-TOF analysis was not attempted on these conjugates because of the mass of KLH (about 1,000,000 Da), but activation using identical conditions was confirmed above (with BSA).

### Example 2

### Fusion Protocol for Hybridoma Production

The methods used in this example were adapted from Lane et al., Methods in Enzymology, 121:183-92 (1986).

Myelomas: Sp2/Ag14 myeloma line used as a fusion partner is available through the American Type Cell Collection. Other similar myeloma lines can also be used for fusions. Grow in HY medium supplemented with 2 mM L-glutamine, 0.15 mg/ml oxaloacetate, 0.05 mg/ml sodium pyruvate, 8.2 ug/ml insulin and serum without antibiotic. Test supernatant for mycoplasma contamination. Keep density between 2×10⁵ and 10⁶ cells/ml; viability should be better than 95%. Once a year subject myelomas to 8-azaguanine selection.

PEG: Different lots of PEG have different fusion efficiency and toxicity. Fusion tested PEG from Sigma or other reliable suppliers can also be used. Fusion PEG solution is preferably made up of 50% PEG, 5% DMSO, 45% serum free medium or buffered saline, sterilized by autoclaving.

Medium: The medium used for fusions and cloning, is the following:

70% HY medium (90% DMEM high glucose 10% NCTC 135)

20% Fetal bovine serum (tested for cloning efficiency of Sp2 myelomas)

2% L-glutamine (200 mM stock)

1% OPI (100× stock from Sigma 05003, stock contains 15 mg/ml oxaloacetate, 5 mg/ml sodium pyruvate, 0.82 mg/ml insulin)

5% Origen (Hybridoma growth supplement, Igen)

1% Hypoxanthine (136 mg/ml stock)

1% Azaserine (10 mg/ml stock; used for selection only)

1% Penicillin/Streptomycin Hypoxanthine/azaserine selection is preferred to HAT selection, since it avoids the high concentrations of thymidine, which can boarder toxic levels and encourages mycoplasma growth. (Foung, et al., PNAS, 79:7484-88 (1982)).

Fusion Procedure:

Bleed mouse and dissect spleen. Mash spleen through a sterile Collector sieve and wash out splenic lymphocytes in 7-15 ml of HY with L-glu and 10% serum.

Pellet cells and suspend in 5 ml ice-cold 0.17 M NH₄Cl (pH 7.5), incubate on ice for 8 min to lyse erythrocytes. Add 10 ml serum-free HY medium, take sample to count, pellet cells, and suspend in serum-free HY medium. Typical yield is 5×10⁷−2×10⁸ lymphocytes with 100% viability.

If in vitro stimulation of lymphocytes is being performed:

Suspend in fusion medium (without azaserine and hypoxanthine) at 10⁷ cells/ml

Add sterile, soluble antigen at 1 μg/ml, and adjuvant peptide (Sigma A9519) at 20 μg/ml

Culture cells for 4 days at 37° C. with 8% CO₂

Count cells again and perform fusion with Sp2 cells according to fusion protocol below. Typically, number of viable lymphocytes will be ˜50% of starting number, but many more blast cells will be visible under the microscope.

Count Sp2 cells, pellet 6×10⁷ Sp2's per 10⁸ splenic lymphocytes, suspend in serum-free HY medium.

Combine Sp2's and lymphocytes in 50 ml conical tube, top off with serum-free HY medium, pellet gently (350 g, 10 min). Pour off supernatant, suspend pellet in residual medium by tapping.

Warm cells and PEG to 37° C., and mix PEG well. Bring cells and PEG into sterile hood in a beaker containing 37° C. water. Have a timer ready (and an assistant to call out times, if desired). Have a 1 ml and a 10 ml pipet ready.

Take up 1 ml of PEG, drip onto cells over 30 seconds with mixing, keeping cells in warm beaker as much as possible. Over the next 15 seconds change to the 10 ml pipet and fill it with 12 ml serum-free HY medium. At 45 seconds from start of fusion, drip 3 ml of medium over 30 seconds with mixing, then drip the remaining 9 ml over the next 30 second period with mixing. Fill the centrifuge tube with medium over the next 30 seconds. The following table shows the steps and times:

| Time | Step | Duration (in seconds) |
|---|---|---|
| 0:00 | Start adding 1 ml fusion mix | 30 |
| 0:30 | Change to 10 ml pipet and fill with | 15 |
| | 12 ml medium | |
| 0:45 | Start adding 3 ml medium 30 | |
| 1:15 | Start adding 9 ml medium 30 | |
| 1:45 | Start adding 36 ml of medium | 30 |

Allow the suspension to stand at room temp for 8 min, then at 37° C. for 2 min. Pellet cells gently (200 g, 10 min), pour off supernatant, suspend pellet in residual medium by tapping.

Pour some complete medium (with azaserine) into tube, transfer to a bottle by pouring. Bring total volume of complete medium to desired level. Suspending fusion products of 10⁸ lymphocytes in 200 ml and plating 0.13 ml per well into 24 96-well plates yields 70-80% of wells with one or more clones. You can distribute cells into wells by taking them up gently into a 5 ml pipet and dripping 2 drops per well, or by pouring the suspension into a sterile multipipettor trough, and using an 8 or 12 channel multipipettor to transfer from the trough to microtiter plates. The cell suspension should be mixed periodically to have uniform distribution. Use wide bore tips for the multipipettor.

Incubate plates in a humidified incubator with 8% CO₂, score wells for number of clones on day 5-6 (count number of clones in one row of each plate).

Feed wells with 0.13 ml (or two drops) complete medium (without azaserine) on day 7. You should be able to start harvesting yellow supernatants around day 10-12. * Clone positive wells immediately. Limiting dilution in complete medium (without azaserine) works well.

Subcloning:

Suspend contents (˜0.2 ml) of a crowded but healthy well (from 96 well plate, estimated cell density 10⁶ cells/ml) into a 24 well plate well with 1 ml medium. This is for expanding the clone, and gives a density of 2×10⁵ cells/ml. If necessary, confirm cell density with a hemacytometer count.

From this new well, do three 1:10 dilutions (e.g. 0.2 ml+1.8 ml), to achieve a density of 200 cells/ml. Dilute again 1.3 ml+11.7 ml, to get 13 ml at 20 cells/ml. Distribute 6.5 ml of this dilution into 1/2 plate, 48 wells, 0.13 ml/well. Add 6.5 ml medium to remaining cells, to get 10 cell/ml density. Distribute 6.5 ml into remaining 1/2 plate, 48 wells, 0.13 ml/well.

Score and record clone # in each well on days 4-6, feed with 0.1 ml on day 7, test all wells (or wells containing clones) by ELISA. From each parental line keep 2-4 subclones that were scored as single clones, and are ELISA positive. If ELISA positives are all multiclonal, subclone again. Grow parental line and each subclone to two 24 wells, harvest 5 ml supernatant, and freeze two vials each.

### Example 3

### Biotinylated Peptide ELISA Protocol for Hybridoma Screening

Reagents Needed:

a) Pierce 15124 Reacti-Bind Streptavidin plates. Alternatively, make your own streptavidin plates by incubating Nunc Maxisorb plates with 10 μg/ml streptavidin in pH 9.5 bicarbonate buffer overnight at 4C. b) PBS/tween Wash Buffer. PBS without Ca⁺² or MG⁺² ions, and With 0.05% Tween 20 (polyoxyethylene sorbitan monolaurate) added. c) Blocking Solution. 0.1% Milk in PBS/Tween wash buffer. This should also be used to dilute the secondary antibody, and may be used for the primary Ab if it is a purified concentrate. d) Secondary Antibody (Affinity purified goat anti-mouse HRP conjugate Jackson 115-035-003 or equivalent) diluted in blocking solution. Dilution factor will vary from lot to lot, usually 1/7500. e) Peroxidase Substrate (TMB from KPL cat # 50-76-04).

Procedure:

1. Make a 10 μg/ml solution of biotinylated peptide antigen in PBS. Rehydrate secondary antigen plates with PBS/Tween for 10 min.

2. Flick plates dry (avoid complete drying of the plates in between all washes; it is best to leave a film of PBS behind, and refill plates with the next reagent before that film dries), and add 100 μl (to conserve antigen, lower peptide concentrations may be used and volumes may be reduced to 50 μl) of antigen solution to each well. Allow antigen to bind to the plates for 1 hour at room temperature. Plates may be stored at −20° C. with antigen until ready to use.

3. Remove antigen (for removal of antigen and washes flick the plates into the sink). Fill wells with blocking solution. Incubate 1 hour at room temperature. Longer blocking or using higher milk concentrations will reduce background.

4. Wash once with PBS/Tween wash buffer.

5. Add 95 μl (or, if conserving antigen, reduce this amount as well) of primary Ab to each well. Let stand at room temperature for 1 hour.

6. Wash 3 times with PBS/tween (3×10 minutes)

7. Re-block for 10 min

8. Add secondary antibody diluted in blocker, at slightly below the volume of the primary Ab (90 μl), incubate for 30 min

9. Wash 3 times with PBS/tween (10 minutes each)

10. Add 200 μl/well of TMB substrate, read at different times between 5 min and ½ hour at 650 nm. End point should be when negative controls are <0.1 OD, and positive controls are >1.0 OD.

### Example 4

### Conjugated Peptide ELISA Protocol for Hybridoma Screening

Reagents Needed:

a) pH 9.6 Buffer. 50 mM sodium carbonate/bicarbonate buffer in 1 liter is made by combining 2.93 g NaHCO₃ and 1.59 g Na₂CO₃. This works well with most antigens. Other pH binding solutions may be used, depending on the nature of the antigen. b) PBS/Tween wash buffer. As per Example 3. c) Blocking Solution—1% Milk in PBS/Tween wash buffer. This should also be used to dilute the secondary antibody, and may be used for the primary Ab if it is being diluted from a concentrate. Hybridoma supernatants contain serum, which acts as a blocker. For peptide antigens weaker blockers are recommended. This could be 0.1% milk, 0.5-3% BSA, or no blocker at all. d) Secondary Antibody. Anti-mouse HRP conjugate (e.g. Roche #605 250 or other commercial sources of affinity purified secondary antibody) diluted in blocking solution. Dilution factor will vary from lot to lot. e) ABTS. (2,2′-azino-di-[3-ethyl-benzthio-line sulfonate] available through Roche cat# 1112 422) 50 mg+50 ml ABTS buffer (#1204 530). f) Nunc Maxisorb or other ELISA plates (Becton-Dickinson Probind, or polycarbonate plates).

Procedure 1. Make a 1-10 μg/ml solution of protein or peptide antigen in the pH 9.6 Buffer. During initial development of the ELISA different concentrations can be tried to determine minimum amount needed for good signal.

2. Add 100 μl (or 50 μl if conserving on antigen) of antigen solution to each well. Allow antigen to bind plates overnight at +4° C. Alternatively, and depending on the nature of different antigens, antigen may be allowed to bind for 2 hour at room temperature. Plates may be stored at −20° C. with antigen in the well.

3. Remove antigen (for removal of antigen and washes flick the plates into the sink). Fill the wells with blocking solution. Block for 1 hr at room temp. Depending on antigen, plates also may be stored with blocking solution. For stronger blocking use higher concentration of blocker and/or longer blocking time.

4. Before use, thaw plates. Wash once with PBS/Tween wash buffer.

5. Add primary Ab, at slightly below the volume of antigen, to each well. Let stand at room temp. for 1 hr.

6. Wash 3 times with PBS/tween (3×10 minutes). Re-block for 10 min.

7. Add secondary antibody, at slightly below the volume of the primary Ab, and incubate for 30 min.

8. Wash 3 times with PBS/tween (10 minutes each).

9. Add 200 μl/well of ABTS solution, read between 5 min and ½ hour at 405 nm Molecular Devices SpectraMax plate reader or comparable reader.

NOTE: Avoid drying of the plates in between all washes.

### Example 5

### Antibody Characteristics

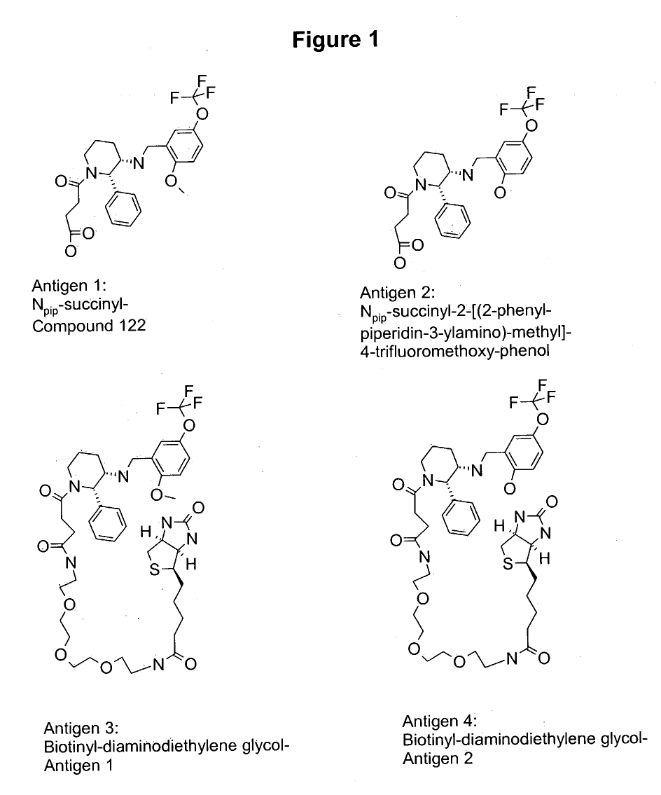

Antibodies specific to Compound 122 and it's desmethyl metabolite (Antigen 2) were generated to be used as reagents in the assay. FIG. 1 shows characteristics of 4 monoclonals generated against Compound 122 and 4 against the desmethyl metabolite. Three were utilized in assay development (9E2.C5, 5D5.D9 and 1E8.B9).

TABLE 1
Preliminary Hybridoma Screens

| | First Screen | | | Second Screen | | |
|---|---|---|---|---|---|---|
| Clone | Compound 122 | Pos Cntrl | Neg Cntrl | Compound 122 | Antigen 2 | Plastic |
| 9E2.C5 (BSA) | .750 | .372 | .060 | .871 | .107 .057 | |
| 5D5.D9(KLH) | .932 | .329 | .068 | .500 | .056 | .057 |
| 8B9.F7(Biot) | .985 | .577 | .069 | .458 | .909 | .090 |
| 12B8.D3(Biot) | .365 | .228 | .054 | .184 | .154 | .081 |
| | First Screen | | | Second Screen | | |
| Clone | Compound 122 | Pos Cntrl | Neg Cntrl | Antigen 2 | Compound 122 | Plastic |
| 1E8.B9 (Biot) | 1.587 | .843 | .058 | 204 | .075 | .053 |
| 8E3.F5 (Biot) | .419 | .556 | .069 | .341 | .058 | .053 |
| 8H8.C11 (Biot) | .520 | .510 | .059 | .346 | .056 | .053 |
| 9E6.C8 (Biot) | .553 | 1.378 | .054 | .459 | .077 | .088 |

5D5.D9 showed good affinity to the parent with little cross-reactivity to metabolite while 1E8.B9 showed good affinity to the metabolite with little cross-reactivity to the parent.

### Example 6

### Lateral Flow Protocol

Small amounts of body fluid will be directly applied to a sample wicking pad (AccuWik) and sample drawn by capillary flow through a conjugate pad containing immobilized detector. In the competitive assay format, the detector reagent consists of colored latex or colloidal gold labeled compound. In the direct assay format, detector reagent consists of colored latex or colloidal gold-labeled antibody. The second mobile agent, a control substance conjugated with a different colored latex or gold sol, will be present in the conjugate pad. The fluid front migrates by capillary action through the nitrocellulose membrane towards the capture region containing permanently immobilized capture reagents. In the competitive format, capture reagent can be antibody if detector is labeled compound or capture reagent can be labeled compound if the detector reagent is color-labeled antibody. In the direct-sandwich assay, capture reagent is antibody. In the competitive format, the absence of a colored symbol (FIG. 2) would indicate high levels of compound. In the direct format, the presence of a colored symbol (FIG. 2) would indicate high levels of compound. A second capture region containing antibodies to the control detector reagent placed downstream of the compound capture reagent indicates proper function of device and also serves as indicator for end-of-assay read.

### Example 7

### Cross-Validation and Direct Measurements of Compound 122 in Saliva

1. Solutions

Stock solutions of Compound 122-HCl salt and internal standard 2-difluoromethoxy-5-trifluoromethoxy-benzyl)-(2-phenyl-piperidin-3-yl)-amine] HCl salt (hereinafter referred to as IS, for internal standard) were prepared at concentrations of 100 μg/ml in 1:1 Methanol/Water and stored at −20° C. The Compound 122 stock solution was found to be stable over a 93 day period. The stability of the Compound 122 stock solution was determined by injecting 10 μl aliquots (diluted 1000:1) onto the HPLC/MS/MS system described below and comparing the response with that of a freshly prepared stock solution. Serial dilutions of the stock solutions were prepared in 1:1 Methanol/Water as needed. Standard curve samples were freshly prepared using serial dilutions of stock so that equivalent additions produced 0.1, 0.2, 0.5, 2, 10, and 50 ng/ml solutions in Control Human Saliva (CHS). Quality control samples were prepared from a different stock solution similarly at 0.15, 5, and 40 ng/ml in CHS.

2. Sample Preparation

Aliquots (50 μl) of Human saliva, 200 μl of 0.1% Ammonium Hydroxide and 50 μl of 2.0 ng/ml IS were added, in sequence, to one well of a 96 well block. The plate was centrifuged briefly to consolidate the above contents and placed onto a TomTec Quadra 96. An automatic program was used that performs the following steps: conditions a Waters brand 10 mg Oasis HLB plate with 100 μl Methanol and then 200 μl 50/50 Methanol/Water (containing 2% v/v Ammonium Hydroxide), transfers the 300 μl of sample to the plate, washes the plate with 400 μl of 50/50 Methanol/Water (containing 2% v/v Ammonium Hydroxide) and then elutes the drug and IS into a clean 96 deep well block with 200 μl of 70/30 Methanol/Water (containing 2% v/v Acetic Acid). The eluent was isolated, evaporated to dryness under a stream of Nitrogen, and reconstituted in 100 μl 60/40 Methanol/10 mM Ammonium Acetate (both containing 0.05% Formic Acid) and vortexed for approximately 30 seconds. The plate was then centrifuged for ˜1 minute at about 3000 RPM. Injection volumes introduced into HPLC system were typically 10 pl.

3. HPLC System

The mobile phase was a binary mixture (60/40) of Methanol and 10 mM Ammonium Acetate (both containing 0.05% Formic Acid). The analytical column was a Phenomenex LUNA Phenyl Hexyl, 5μ, 2.00×50 mm LC/MS column preceded by a 2.0 micron stainless steel precolumn filter. A Hewlett Packard 1100 series quaternary pump was used and a mobile phase flow rate of 0.30 ml/min was maintained. A CTC Analytics (LEAP) HTS PAL autosampler injected the 10 μl sample aliquots onto the column at approximately 3 minute intervals. Under these HPLC conditions, both Compound 122 and the IS had elution times of approximately 85 seconds.

4. Mass Spectrometry

The analysis was performed on a Perkin Elmer SCIEX API 3000 triple quadrupole mass spectrometer operated in the positive ion mode. The effluent from the HPLC column was directly introduced into the TurbolonSpray ion source, which was operated at 1500V with a temperature of 375° C. and 6 L/sec nitrogen gas. Nitrogen nebulizer gas was set to 10 and curtain gas was set to 9. Analyte and IS responses were measured using multiple reaction monitoring (MRM). Protonated molecular ions for drug (m/z 381.4), and I.S. (m/z 417.1) were dissociated by collision with nitrogen. Collision gas (CAD) was set to 5 and a collision energy of 32 eV was used. Product ions at m/z 160.0 were monitored for both drug and IS runs. This assay was used to measure levels of Compound 122 in saliva in the samples collected as described in Example 8.

### Example 8

### Compound 122 Salivary Concentrations in Healthy Human Subjects (CYP2D6 Extensive and Poor Metabolizers) After Oral Administration of 10, 30, and 100 mg With and Without Coadministration of Paroxetine

The objective of this study was to determine the saliva concentrations of Compound 122 after oral administration to healthy human subjects including CYP2D6 extensive and poor metabolizers (EMs and PMs) and subjects coadministered paroxetine, a CYP2D6 inactivator.

Subjects were divided into three groups of six individuals: CYP2D6 extensive metabolizers (including one “intermediate” metabolizer), CYP2D6 extensive metabolizers receiving concurrent paroxetine as an inhibitor of CYP2D6 (also including one intermediate metabolizer), and CYP2D6 poor metabolizers. Subjects received Compound 122 HCl salt in five study legs as follows: 10 mg q.d. for five days, followed by 10 mg b.i.d. for five days, followed by 30 mg q.d. for five days, followed by 30 mg b.i.d. for five days, and finally 100 mg q.d. for five days. Saliva samples were collected on the first day of each study leg.

Saliva was assayed for Compound 122 using a validated HPLC-MS/MS assay (see Example 6). Samples were subjected to a solid phase extraction procedure followed by chromatography and analysis on a Sciex API3000 tandem quadrupole mass spectrometer.

Mean values were calculated in those cases in which half or greater of the individual values were >LLOQ. A value of zero was used in those cases wherein concentrations were <LLOQ. Saliva Compound 122 AUC (0-24 hr) values were calculated using the linear trapezoid method. Cav is defined by AUC (0-24 hr)/24.

A comparison of mean concentration data in the three subject groups (CYP2D6 EM subjects, PM subjects, and EM subjects receiving concomitant paroxetine) is in Table 2. Intersubject variability was great within each of the three dosing groups, with %CV values generally greater for EM subjects than PM subjects. Exposure values for the IM subjects were not markedly different from EM subjects within the same dose group.

TABLE 2
MEAN SALIVA CONCENTRATIONS OF
COMPOUND 122 AFTER ORAL ADMINISTRATION
TO HEALTHY HUMAN SUBJECTS

| | | | | | EM + |
|---|---|---|---|---|---|
| | Dose | Time | EM | PM | paroxetine |
| Day | (mg) | (hr) | Mean ± SD | Mean ± SD | Mean ± SD |
| 1 | 10 q.d. | 0 | | | |
| 1 | 10 q.d. | 2 | 0.545 ± 0.518 | 0.517 ± 0.261 | 0.500 ± 0.462 |
| 1 | 10 q.d. | 4 | 0.524 ± 0.293 | 2.232 ± 1.061 | 0.632 ± 0.773 |
| 1 | 10 q.d. | 8 | 0.313 ± 0.164 | 2.308 ± 1.316 | 0.533 ± 0.722 |
| 1 | 10 q.d. | 24 | | 1.054 ± 0.558 | 0.192 ± 0.332 |
| 6 | 10 b.i.d. | 0 | 0.270 ± 0.454 | 2.93 ± 2.45 | 3.21 ± 5.09 |
| 6 | 10 b.i.d. | 2 | 0.541 ± 0.465 | 2.85 ± 2.18 | 1.92 ± 1.83 |
| 6 | 10 b.i.d. | 4 | 0.987 ± 0.915 | 4.69 ± 3.28 | 2.97 ± 2.16 |
| 6 | 10 b.i.d. | 8 | 0.805 ± 1.184 | 3.23 ± 2.80 | 5.68 ± 9.27 |
| 6 | 10 b.i.d. | 24 | 0.418 ± 0.426 | 5.51 ± 4.39 | 2.16 ± 1.27 |
| 11 | 30 q.d. | 0 | 1.16 ± 1.81 | 8.31 ± 6.80 | 3.86 ± 1.57 |
| 11 | 30 q.d. | 2 | 3.07 ± 2.77 | 6.96 ± 3.73 | 5.98 ± 2.90 |
| 11 | 30 q.d. | 4 | 4.03 ± 4.10 | 11.1 ± 8.5 | 9.51 ± 3.07 |
| 11 | 30 q.d. | 8 | 3.18 ± 2.99 | 14.0 ± 9.7 | 6.30 ± 1.69 |
| 11 | 30 q.d. | 24 | 1.92 ± 3.13 | 11.8 ± 9.3 | 5.59 ± 2.28 |
| 16 | 30 b.i.d. | 0 | 2.44 ± 4.17 | 15.0 ± 10.9 | 6.16 ± 4.58 |
| 16 | 30 b.i.d. | 2 | 2.91 ± 2.49 | 11.4 ± 6.5 | 9.11 ± 5.40 |
| 16 | 30 b.i.d. | 4 | 4.57 ± 3.03 | 12.9 ± 7.5 | 10.0 ± 6.0 |
| 16 | 30 b.i.d. | 8 | 3.44 ± 3.67 | 13.3 ± 9.9 | 9.69 ± 4.74 |
| 16 | 30 b.i.d. | 24 | 5.08 ± 7.08 | 21.5 ± 18.4 | 9.67 ± 5.91 |
| 21 | 100 q.d. | 0 | 6.94 ± 6.81 | 32.9 ± 17.1 | 12.2 ± 2.7 |
| 21 | 100 q.d. | 2 | 19.1 ± 16.6 | 23.1 ± 12.1 | 35.5 ± 24.7 |
| 21 | 100 q.d. | 4 | 21.2 ± 13.6 | 46.2 ± 29.1 | 44.1 ± 32.3 |
| 21 | 100 q.d. | 8 | 14.6 ± 8.6 | 41.3 ± 26.2 | 27.2 ± 10.0 |
| 21 | 100 q.d. | 24 | 12.5 ± 15.4 | 46.4 ± 27.1 | 15.7 ± 8.8 |

In general, saliva concentrations of Compound 122 were greatest in PM subjects. Mean salivary AUC (0-24 hr) values in PM subjects ranged from 2.8 to 6.5-fold of those in EM subjects. Greater differences between these two groups were observed at the low doses. Values for EM subjects with concomitant paroxetine were in between the values for EM and PM subjects.

A comparison of mean saliva concentration values at 2, 4, 8, and 24 hr post-dose sampling times (q.d. only) between EM, PM, and EM subjects with paroxetine showed differences between EM and PM subjects were more apparent at later timepoints, and at lower doses. The greatest differences were observed in individual values at the 24 hr timepoint. At the 10 mg dose, only one EM subject had a greater Compound 122 saliva concentration than the PM subject with the lowest concentration. The extent of overlap between the Compound 122 salivary concentrations in the lowest PM subjects and the highest EM subjects increased with increasing dose.

In all three dose groups, the 10-fold increase in dose (10 to 100 mg) yielded greater than 10-fold increases in exposure (59-26- and 62-fold increases in EM, PM, and EM subjects with paroxetine). The increased exposure with oral dosing was more pronounced at the lower range of doses, and was more marked in EM and EM subjects with paroxetine than in PM subjects.

Interpretation:

These results indicate that salivary concentrations of Compound 122 are readily measurable after oral administration of 10-100 mg/day, and that differences can be observed between CYP2D6 EM and PM subjects. In particular, the difference between EM and PM salivary concentrations observed 24 hr post-dose of low doses of Compound 122 (10 or 30 mg) suggests that such a measurement could potentially be used in a non-invasive test to identify CYP2D6 phenotype with regard to this agent. However, distinguishing EM subjects from those also taking paroxetine may not be as readily possible. Multiple dosing of paroxetine is reported to convert CYP2D6 EM subjects to CYP2D6 PM “phenocopies.” For Compound 122 saliva concentrations, paroxetine did cause an increase, however that increase did not match the concentrations observed in CYP2D6 PM subjects.

Salivary drug concentrations should be expected to be reflective of unbound serum concentrations of Compound 122. In early experiments, the unbound fraction of Compound 122 in human serum was measured at fu=0.07. Thus, dividing the salivary concentration by fu should yield a value that is close to the total serum concentration. Interestingly, the dose of 30 mg b.i.d. yielded a mean salivary Cav value of 4.04 ng/mL, which would correspond to a serum Cav of 58 ng/mL. Stronger correlation of salivary concentrations to serum concentrations can be made as corresponding serum pharmacokinetic data become available.

Salivary exposure to Compound 122 increased with increasing dose. Exposures were highly variable, with %CV values typically in excess of 100%.

Overall differences in salivary exposures were observed between CYP2D6 extensive and poor metabolizers. These differences were greatest at the low doses. The differences suggest that a point-of-contact test for Compound 122 salivary concentrations could potentially be utilized to distinguish CYP2D6 EM and PM subjects, and could also be used to assign different Compound 122 doses for these two classes of subjects, if necessary.

Equivalents

While specific embodiments of the subject invention have been discussed, the above specification is illustrative and not restrictive. Many variations of the invention will become apparent to those skilled in the art upon review of this specification. The appended claims should be interpreted by reference to the claims, along With their full scope of equivalents, and the specification, along with such variations.

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

What is claimed is:

1. A method for determining whether a person is a poor metabolizer or an extensive metabolizer of Compound 122, said method comprising the steps of:
a) administering a test dosage of Compound 122 to said person;
b) measuring the concentration of Compound 122 in a saliva sample from said person at a predetermined time period after said administration step; and
c) classifying said person as a poor metabolizer or an extensive metabolizer of Compound 122 based upon the concentration of Compound 122 as measured in said measuring step.

2. The method according to claim 1 wherein said time interval is 4 hours or less.

3. The method according to claim 1 wherein said time interval is 2 hours or less.

4. The method according to claim 1 wherein said time interval is 1 hour or less.

5. The method according to claim 1 wherein said test dosage is administered orally.

6. The method according to claim 1 wherein said test dosage is 30 mg or less.

7. The method according to claim 1 wherein said test dosage is 10 mg or less.

8. The method according to claim 1 wherein said concentration of Compound 122 in the saliva is measured by a lateral flow assay.

9. The method according to claim 1 wherein said concentration of Compound 122 in the saliva is measured using HPLC or mass spectrometry.

10. The method according to claim 1 wherein said person is classified as a poor metabolizer if the concentration of Compound 122 in said person's saliva is greater than 1 ng/ml.

11. The method according to claim 1 wherein said person is classified as a poor metabolizer if the concentration of Compound 122 in said person's saliva is greater than 1 ng/ml between two and three hours after receiving a 10 mg oral test dosage.

12. A method for determining the proper dose of Compound 122 to be given to a patient, said method comprising the steps of:
a) administering a test dosage of Compound 122 to said patient;
b) measuring the concentration of Compound 122 in a saliva sample from said patient at a predetermined time period after said administration step; and
c) determining that said patient requires a low dosage treatment of Compound 122 if said saliva concentration is high, and that said patient requires a standard dosage treatment of Compound 122 if said saliva concentration is low.

13. The method according to claim 12 wherein a low dosage treatment of Compound 122 comprises 10 mg q.d.

14. The method according to claim 12 wherein a standard dosage treatment of Compound 122 comprises 30 mg q.d.

15. The method according to claim 12 wherein said saliva concentration is determined to be high if it is greater than 1 ng/ml when measured from 1 to 4 hours after oral administration of a 10 mg test dosage.

16. The method according to claim 12 wherein said saliva concentration is determined to be low if it is less than 1 ng/ml when measured from 1 to 4 hours after oral administration of a 10 mg test dosage.

17. A device for measuring the salivary concentration of Compound 122, said device comprising:
a) a lateral flow membrane;
b) a saliva application zone on said membrane; and
c) an indicator zone on said membrane spaced laterally from said saliva application zone; wherein immobilized in said indicator zone is an antibody specific for Compound 122, which antibody is labeled in a manner that provides an easily read color change if saliva applied to said saliva application zone contains an adequate concentration of Compound 122.

18. The device according to claim 17 wherein said adequate concentration of Compound 122 is equal to or greater than 1 ng/ml.

19. The device according to claim 17 wherein said label is colored latex particles or colloidal metal.

20. An antibody that specifically binds to Compound 122 wherein said antibody does not specifically bind to naturally occurring metabolites of Compound 122.
